(19) United States
(12) Patent Application Publication
(10) Pub. No.: US 2019/0391106 A1
(43) Pub. Date: Dec. 26, 2019

(54) Organic Nanobiomimetic Memristive/Memcapacitive Devices Ultrasensitive Direct Detect Matrix Metalloproteinase

(71) Applicant: ELLEN TUANYING CHEN, ROCKVILLE, MD (US)

(72) Inventor: ELLEN TUANYING CHEN, ROCKVILLE, MD (US)

(21) Appl. No.: 15/978,102

(22) Filed: May 12, 2018

Related U.S. Application Data
(60) Provisional application No. 62/505,913, filed on May 14, 2017.

(51) Int. Cl. G01N 27/40; G01N 33/573; G01N 27/27; G01N 27/414; G01N 27/30

(52) U.S. Cl. CPC G01N 27/40; G01N 33/573; G01N 27/30; G01N 27/4145; G01N 27/27

(57) ABSTRACT

A dual-functioning electrochemical sensing device has invented for fast, direct ultrasensitive detection of protein, such as attomolar concentration (aM) Matrix Metalloproteinase (MMP) without a procedure of cycteine switch under label-free, probe-free and reagent-free conditions. The invented device comprises of an organic memcapacitive/memristive membrane by self-assembling forming polarized 3D array crossing-nanotube structures on a gold substrate that enables the membrane selectively induction of bio-communication with MMP-2 in the absence of interference from other proteins in human serum specimens, herein aM concentration MMP-2 can be reliably detected by two different methods.

### CROSS-REFERENCE TO RELATED APPLICATIONS

This patent application claims the benefit of U.S. Provisional Patent Application Ser. No. 62/505,913 filed on May 14, 2017. The entire disclosure of the prior Patent Application Ser. No. 662/505,913 is hereby incorporated by reference, as is set forth herein in its entirety.

### FIELD OF THE INVENTION

The present invention relates to the field of electrochemical sensors, in particular, to a device having both characteristics in memristive/memcapacitive for direct reagent-free sensing of attomolar concentration (aM) of Matrix Metalloproteinase (MMP) in biological specimens.

### BACKGROUND OF THE INVENTION

Matrix Metalloproteinase (MMP) is a family of zinc-dependent endopeptidases. The enzymes play a key role in human health for promoting newborn growth, nervous system growth, as well as in promoting various human diseases, such as cancer invasion, osteoarthritis, tissue destruction, diabetes, coronary malfunction, epilepsy and Alzheimer's [1-4]. MMP's major role is to degrade the extracellular matrix as a double-edge sword. MMP-2 has been identified as a critical biomarker for diagnosing, monitoring and predicting multiple types of human diseases [4-9]. However, almost 50 MMP inhibitors in clinical trials failed due to lack of specificity of the inhibitor to MMP [10]. Improving sensor performance in the detection of MMPs in a sub fg/mL level among a wide dynamic range implemented with simplified procedures is a paramount challenge in the traditional enzyme-linked immunosorbant assays (ELISAs) method, labeling florescence method and the nanoparticle electrochemical sensing methods [11-12]. This is because most methods are subject to protein interference and time consuming burdensome procedures that hamper reaching the goals. Our prior experiences in the development of nanostructured biomimetic sensors for direct detection of various biological biomarkers have encouraged us to seek an innovative approach and attempt to attack this problem for direct reagent-free detection of MMP-2 [13-18].

Development of polarized microtubules mimicking nature's microtubules is an increasingly interesting subject in many nanoscale engineering applications [19]. However, utilizing the microtubule mimicking approach to apply to the direct detection of MMP-2 with a reagent-free goal in mind is very challenging, and then the question which follows is how to induce MMP-2 direct biocommunication with the artificial microtubules. Our approach is to build the artificial microtubules with cross-linked organic conductive polymers having multiple chelating imidazole ligands embedded. That enables the polymer ligands to have a strong affinity to coordinate with the zinc ions in the MMP-2. Plus the crossing-bar nanotubules might be favorable in developing a nanostructured memcapacitive/memristive sensor for reagent-free, probe-free direct measurement of MMP-2.

### SUMMARY OF INVENTION

It is an object of the present invention to create an organic nanobiomimetic memristive/memcapacitive devices having sensing function for direct detection of protein molecules, and specifically for MMP without using a probe or denaturing the protein.

It is an object of the present invention to use the innovative memristive/memcapacitive sensors to quantitative detect MMP in attomolar concentration (aM) in a biological specimens without the presence of other protein interference.

It is an object of the present invention to detect MMPs in biological specimens in a 2-4 ms speed without sample preparation or treatment.

It is an object of the present invention to detect MMPs under conditions of reagent-free.

It is a further object of the present invention having the device able to be dual-functions, i.e., be a Chronoamperometric sensor and a voltage sensor.

It is a further object of the present invention to have a Detection of Limits (DOL) reaching orders of magnitude lower than published reports under antibody-free, tracer-free, and reagent-free conditions with simplified procedures.

### BRIEF DESCRIPTION OF THE DRAWINGS

FIG. 1A depicts the AFM 3D image for device in a small 1 μm length scale. FIG. 1B is the cross-section analysis.

FIG. 2 depicts ordered cross nanotubes in a large 5 μm scale AFM image.

FIG. 3. depicts the art model for the proposed polarizable microtubule electron-relay system. The red dot refers to the imidazole receptors in the CD cavity.

FIG. 4(A) Matrix Metalloproteinase Illustrates the hysteresis of the i-V curve of the memristor/memcapacitor in 40 ng/mL MMP-2 with consecutive scan cycles at 200 Hz scan rate.

FIG. 4 (B) depicts the i-V curve in control solution at 200 Hz scan rate.

FIGS. 5A, 5B and 5C represent the plots of normalized current of DET_{red}, DETₒₓ and the MEM peaks vs. 5 scan cycles, respectively.

FIG. 6 (A) depicts a plot of the current density vs. MMP-2 concentration over the range of 2.0×10⁻¹⁷ g/mL to 1.0×10⁻⁷ g/mL with triplicates. FIG. 6 (B) depicts the CA curve profiles from 0.01 ng/mL to 100 ng/mL.

FIG. 7(A) depicts the CA curve profiles from 0.02 fg/mL to 40 fg/mL. The insert is the linear regression curve over the same MMP2 range. FIG. 7(B) depicts a linear calibration plot of the current density vs. MMP-2 concentration range from 0.01 ng/mL to 100 ng/mL with triplicates

FIG. 8(A) depicts the DSCPO voltage curves vs. time at 0.25 Hz at ±10 A over 40 ag/mL to 100 ng/mL MMP-2 concentrations against the control samples with each sample run triplicates. FIG. 8 (B) depicts the volumetric energy density vs. MMP-2 concentrations.

FIG. 9 depicts the relationship between energy density of the sensor, MMP-2 concentration and specific capacitance using the voltage method.

FIG. 10A depicts the energy density vs. applied potential curves in the control PBS solution at 400 Hz scan rate of 5 consecutive scan cycles. FIG. 10B depicts the energy density vs. applied potential curves in the presence of 40 ng/mL MMP-2 in 5 consecutive scan at 200 Hz san rate. FIG. 10C depicts the comparison of capacitance vs. applied potential in the control PBS and FIG. 10D depicts the curves in the presence of 40 ng/mL MMP-2.

### DETAILED DESCRIPTION OF THE INVENTION

### Example 1—Fabrication of the Nanostructured Self-Assembling Membrane (SAM) Gold Memristive/Memcapacitive Chips

The nanostructured biomimetic SAM was freshly prepared by forming cross linked conductive polymers from triacetyl-β-cyclodextrin (TCD), polyethylene glycol diglycidyl ether (PEG), poly(4-vinylpyridine) (PVP) and bis-substituted dimethyl-β-cyclodextrin (bM-β-DMCD) in a self-assembling manner on gold chips with appropriate propositions of the mixture. The polymer mixture was incubated at 80° C. for 2 hrs before injecting it on the chip. After the injection, the chips were incubated for 96 hrs at 37° C., then reincubated again for 2 hrs after washing the chip with high purity water. The procedures of synthesis and characterization of bM-β-DMCD were based on the published literature [20]. MMP-2 enzyme was purchased from Ana Spec (Freemont, Calif.).

### Example 2—Characterization of the Biomimetic Microtubule Membrane

The morphology of the AU/SAM was characterized using an Atomic Force Microscope (AFM) (model Dimension Edge AFM, Bruker, MA). Data collected in TappingMode using silicon probes with 5-10 nm tip radius and −300 kHz resonance frequency (Probe mode TESPA-V2, Bruker, MA).

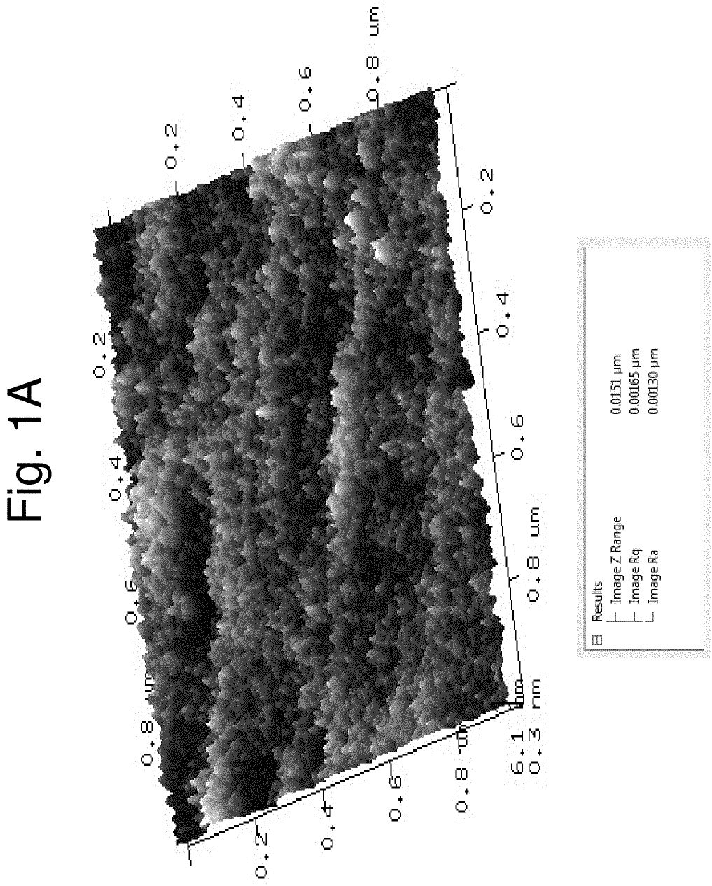

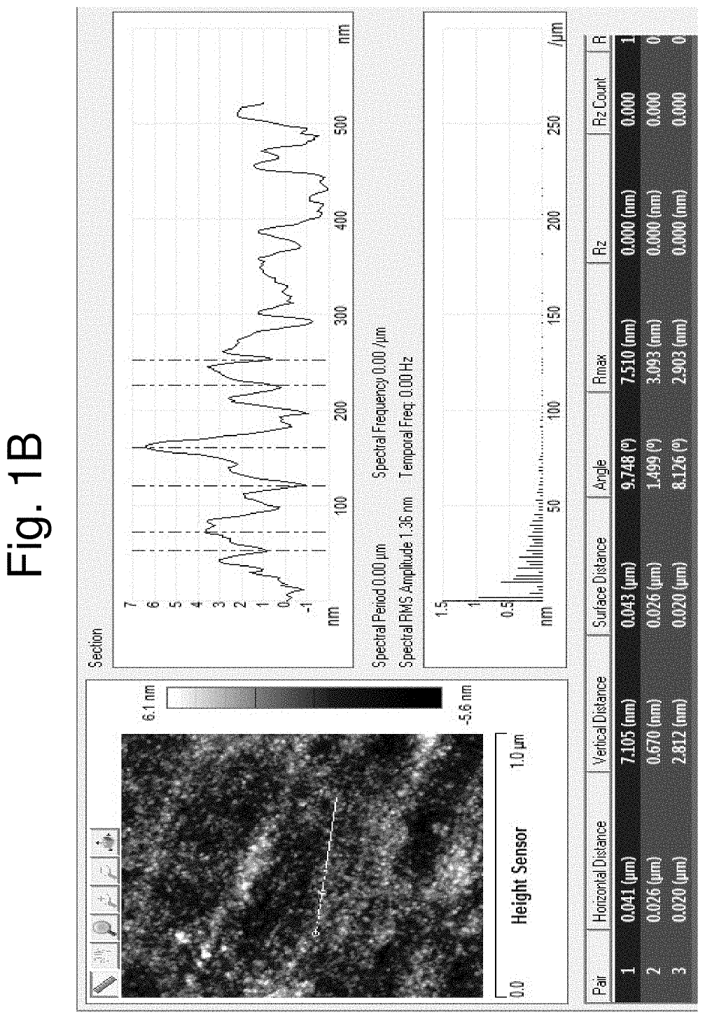

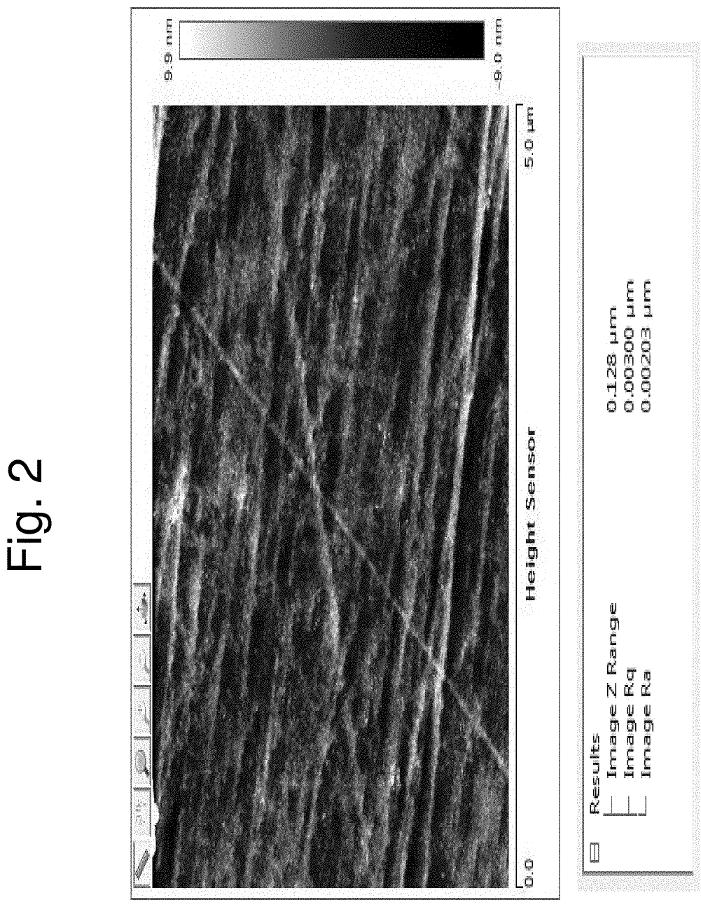

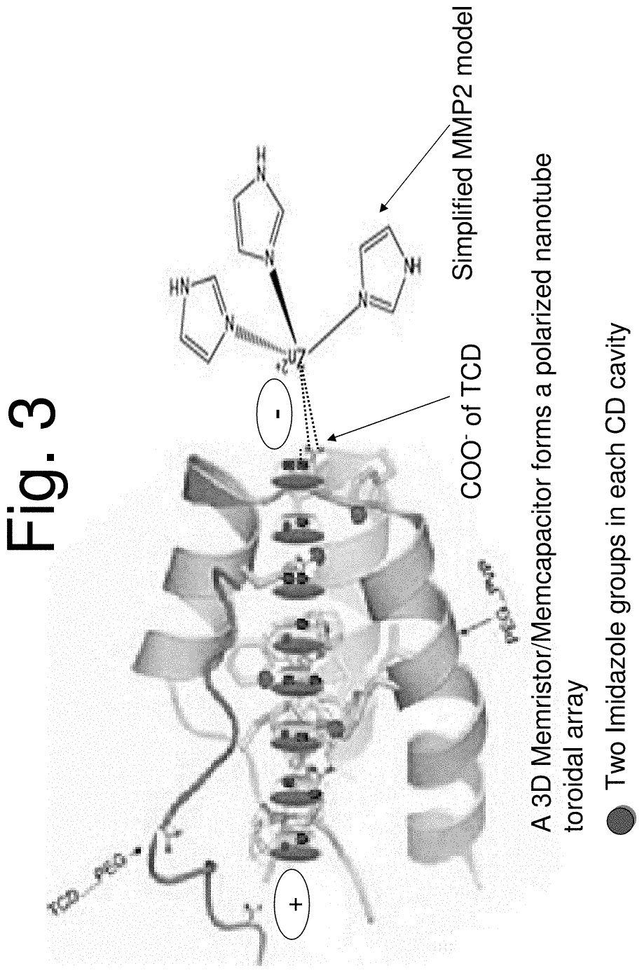

FIG. 1A illustrates the 3D structure of the membrane with an array of vertical nanopillars with crossing-bars in multiple layers in small scale with z value 15.1 nm, R_{q} 1.7 nm and Rₐ 1.3 nm. The amplitude channel shows changes in slopes and edges of features seen in height image, similar to a derivative of the Height channel. FIG. 1B depicts the cross-section analysis of FIG. 1A. FIG. 2 shows the AFM well-ordered crossing-nanotube image in large scale. The proposed direct electro-transfer (DET) relay mechanism for detecting MMP-2 was depicted in an art model in FIG. 3. The right hand side is the simplified MMP model, and the induced direct bio-communication was shown through the zinc ion coordinating with both of the COO⁻ of TCD and the receptor groups of two imidazole in bm-β-DMCD cavity, i.e., by the coordination geometry, proton and electron transfers and the displacement of water molecules which formed the long electron-relay chain based on a favorable low AG [22-23]. The advantage of the sensor SAM is that it turns the inhibitory nature of the MMP-2 coordination complex as a potential cancer treatment drug into an agent for stimulating MMP-2 expression and forms polarizable microtubules shown from FIG. 1A, FIG. 1B to FIG. 3.

### Example 3—Evaluation of the Microtubule Polarizable Behavior During the Coordination Formation

Evaluations of the formation of a coordination complex between the MMP-2 and the ligands of the biomimetic membrane were based on a model mechanism proposed in FIG. 3. Two methods were used for the evaluations: (1) A cyclic voltammetry (CV) method was used to compare the dynamic rate constant results of direct electron-relay peaks and the MEM peaks vs. consecutive scan cycles (5) with or without MMP-2 at 200 mV/s scan rate at room temperature at pH 7.4 PBS. MMP-2 concentration is 40 ng/mL. (2) A comparison of Michaelis-menten constant (kₘ) results using curves obtained from a chronoamperometric method (CA) described in the following section.

Comparing the Rate Constant by the CV Method.

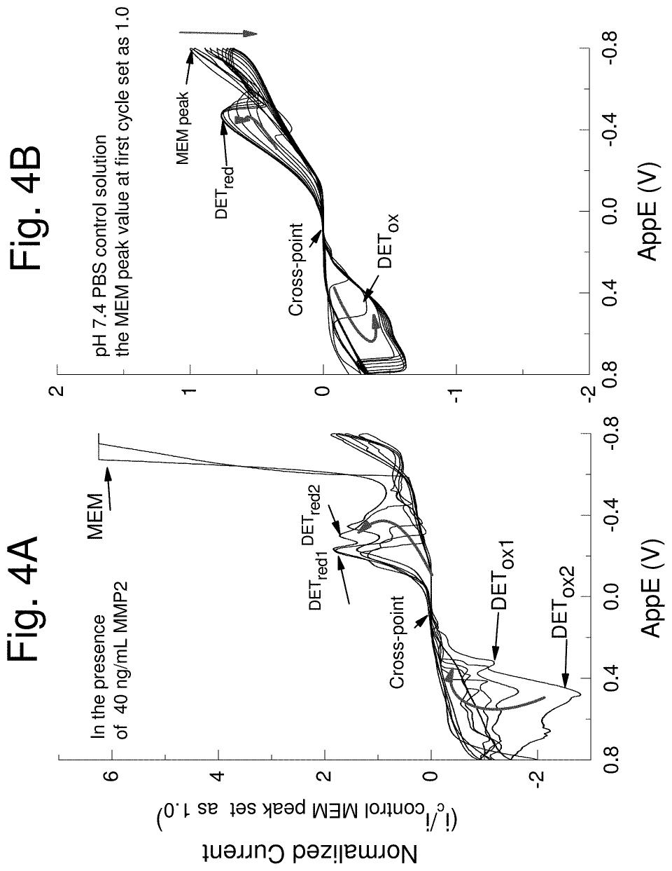

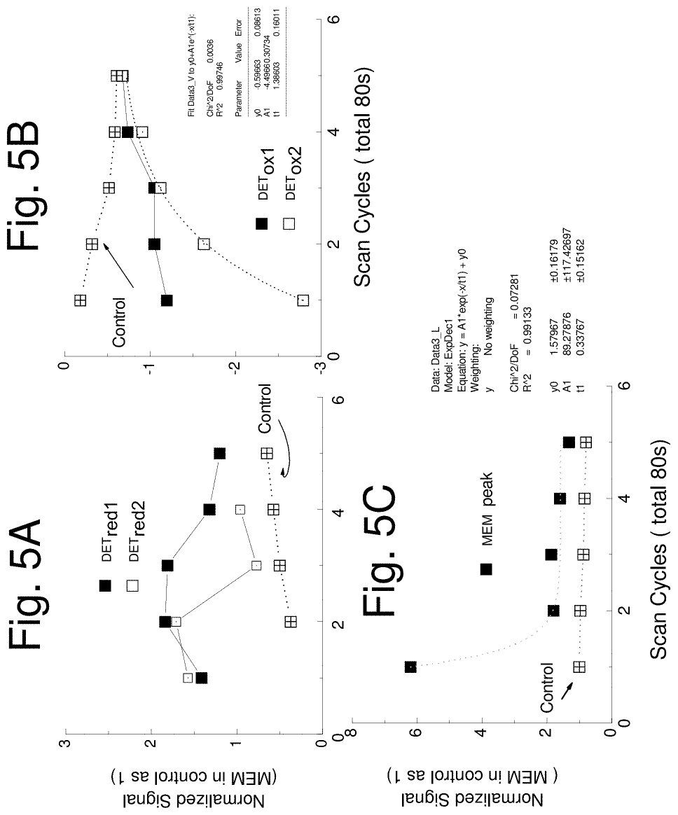

Memristor/memcapacitor exhibits not only hysteretic charge-voltage and capacitance-voltage curves but also negative and diverging capacitance within certain ranges of the field [25]. FIG. 4(B)'s i-V hysteresis curve demonstrated with a switch point at the origin (0, 0) under consecutive scans at 200 Hz in PBS solution compared with that of having 40 ng/mL MMP-2 shown in FIG. 4(A). The increased positive and negative nonlinear potential movements of the DET_{red} and DETₒₓ peaks from the origin demonstrate there is a bidirectional polarizable forces exist in the microtubules as the scan cycles increased. FIGS. 5A, B and C represent the plots of normalized current of DET_{red}, DETₒₓ and the MEM peaks vs. scan cycles, respectively. Table 1 compares the DET peak and the MEM peak rate constant results vs. scan cycles for with or without MMP-2. The results show the sensor interactions with MMP-2 have drastically broken the balanced, bidirectional, polarizable direct electron-relay and hole hopping, into a more powerful asymmetric system. The rate constant and the amplitude of the MEM peak with MMP-2 increased 123 and 35.6-fold compared with the control, respectively. This demonstrates the charge of zinc ion coordinating with the gate ligand COO⁻ first at the sensor membrane with the rate constant and the amplitude of DETₒₓ₂ increased to 1.8 and 5.3-fold compared with the control indicating electron-relaying has taken place from COO⁻ to the first imidazole's proton transfer as the second step. Then the third step is the DET_{red1} peak with MMP-2 which increased the rate constant and the amplitude to 2 and 1.2-fold when coordinating with the second imidazole's free pair electrons compared with the control, respectively. Herein, directly detecting MMP-2 without using neither an antibody nor any other agents to activate MMP-2 in order to have a “cysteine switch” made possible which is impossible according to conventional teaching [11-12, 26].

TABLE 1
Evaluation of the rate constant of monitored normalized peaks
signal strength with or without MMP2 vs. scan
cycles using the memristor/memcapacitor

| | MMP2 | Rate^{a} | | | | |
|---|---|---|---|---|---|---|
| Type | (40 ng/mL) | (s⁻¹) | Y₀ | A₁ | r | Chi\hat{ }2/DOF |
| DET_{red} | No | 0.019 | 0.83 | −0.84 | 0.998 | 0.0002 |
| DETₒₓ | No | 0.025 | −0.74 | 0.85 | 0.986 | 0.0019 |
| MEM | No | 0.002 | −1.44 | 2.51 | 0.989 | 0.0003 |
| DET_{red1} | yes | 0.04 ^{b} | — | 1.02 | 0.877 | — |
| DET_{red2} | yes | — | — | — | — | — |
| DETₒₓ₁ | Yes | 0.008 ^{c} | −1.35 | — | 0.955 | — |
| DETₒₓ₂ | yes | 0.045 | −0.60 | −4.50 | 0.999 | 0.0036 |
| MEM | yes | 0.185 | 1.58 | 9.3 | 0.996 | 0.07 |

^{a}refers to the first order rate constant model for exponential decay of the normalized different types of peaks strength monitored vs. time in second. All other parameters of Y₀, A₁ are referring to the normalized signal strength per scan cycle. For the control samples, the consecutive 5 scan cycles were at 200 mv/s and used to compare with that in the presence of 40 ng/mL MMP2 samples at the same experimental conditions.
^{b} For DET_{red1} with MMP2, curve fitted with a polynomial model Y = A + B1*X + B2*X\hat{ }2.
^{c} for the DETₒₓ₁ it was fitted with a linear model.

Comparing the Kₘ Constant.

For comparing the Km results of the ligands of the sensor membrane affiliated with the MMP-2, Lineweaver-Burke plots were constructed. The Km value is 6.75 pM over 7.0×10⁻¹³ to 1.4×10⁻⁹ M, which is orders of magnitudes stronger complexation than reported MMP-2's Km value for type 1 collagen of 8.5 μM [27-28]. The MMP-2 concentration is between 2×10⁻¹⁷ to 8.0×10⁻¹⁶M, K_{c} value is 1.6×10⁷/s and the K_{c}/Kₘ=6.4×10¹⁸ s⁻¹·M⁻¹.

### Example 4—Quantitation of MMP-2

Quantitation of MMP-2 was conducted in two methods: the CA method and the Double Step Chronopotentiometry (DSCPO) method. The data were acquired at room temperature under fixed applied potentials for the CA method with 4 MHz data rate in MMP-2 final concentrations ranging from 2.0×10⁻¹⁷ g/mL to 1.0×10⁻⁷ g/mL with triplicates compared with pH 7.4 PBS controls. Curves presented were after taken an absolution for better visualization. Fixed ±10 nA and 4 s step time was used with 1 KHz data rate for the DSCPO method with similar MMP-2 concentration ranges with samples run triplicate. MMP-2 samples were freshly prepared. Before the measurements, the standards samples were incubated at 37° C. for 2 hours. The preliminary applications were to detect the MMP-2 activities present in the NIST SRM 965A reference human serum samples with known hypo-, normal and hyperglycemia concentrations, respectively. An electrochemical work station was used (Epsilon, BASi, IN) with a software package from BASi. Origin Pro 2016 (Origin Lab Corp., MA) was used for all statistical data analysis and figure plotting.

Quantitation of MMP-2 by the CA Method.

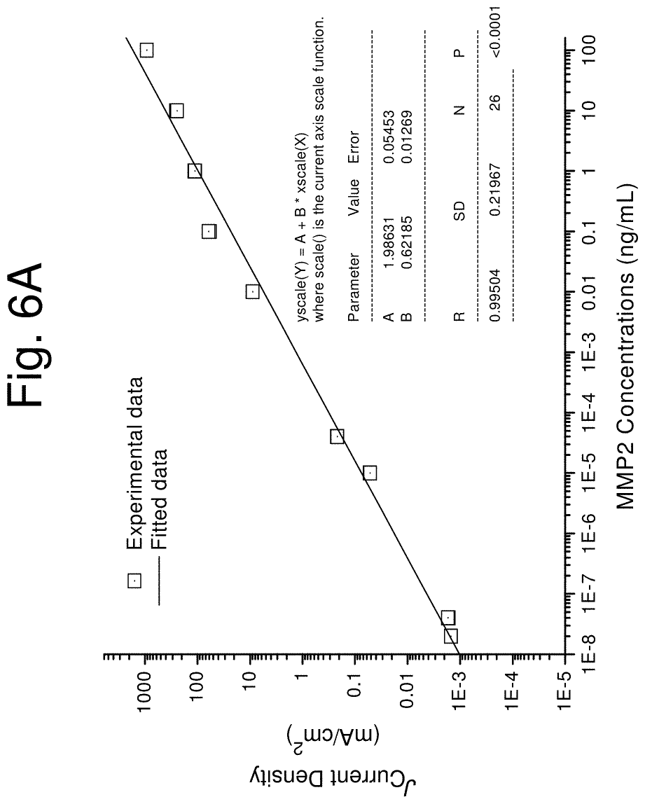

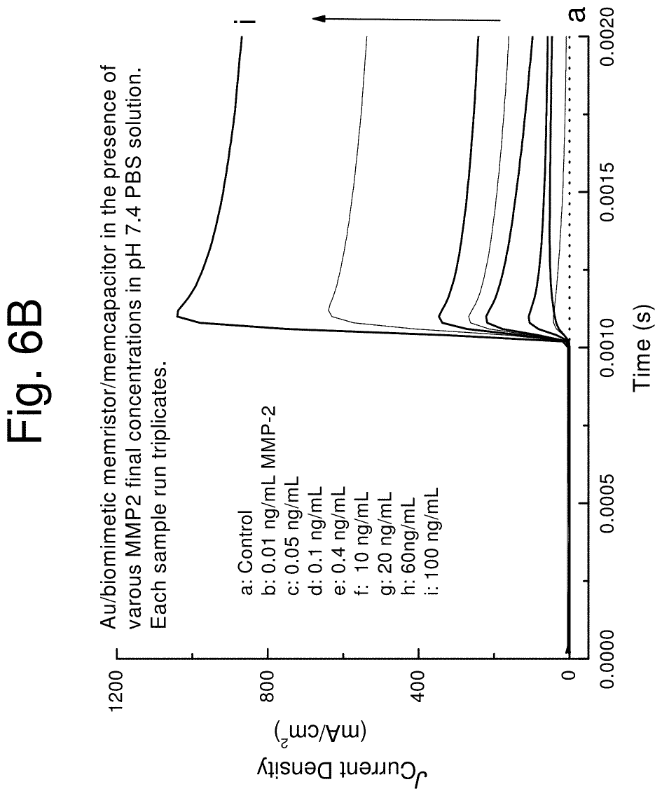

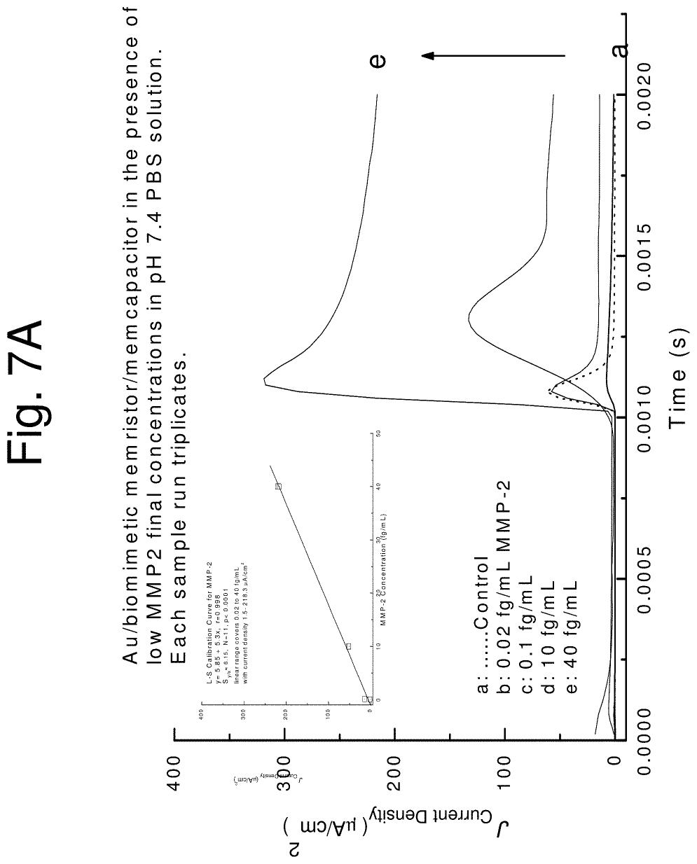

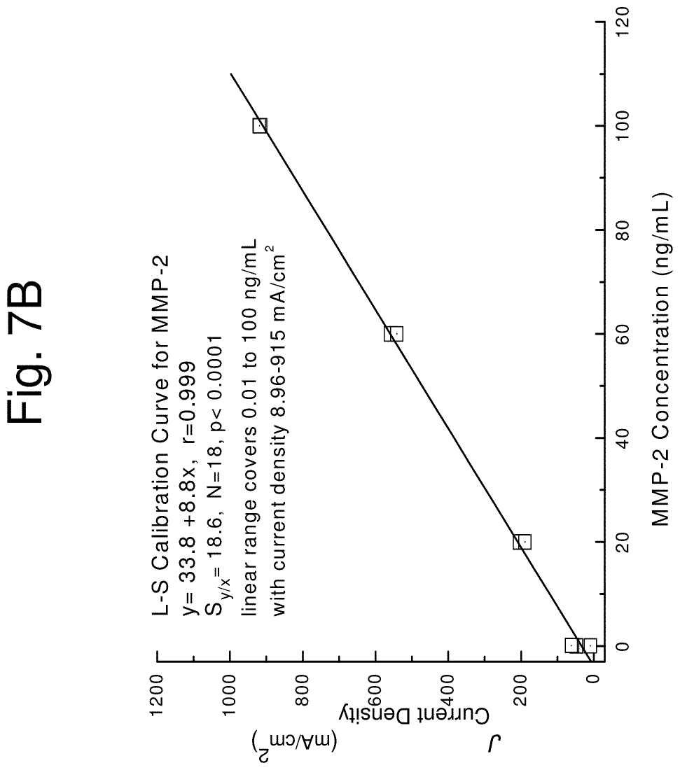

FIG. 6 (A) depicts a plot of current density vs. MMP-2 concentration over the range of 0.02 fg/mL to 100 ng/mL. The CA method has a Detection of Limits (DOL) value of 8.67×10⁻¹⁸ g/mL in PBS solution related to current density between 1.47 μA/cm² and 919.2 mA/cm² over MMP2 concentration between 20 ag/mL to 100 ng/mL with a Relative Pooled Standard Deviation 1.4% (n=26). FIG. 6(B) depicts the CA curve profiles from 0.01 ng/mL to 100 ng/mL. FIG. 7(L) depicts the CA curve profiles from 0.02 fg/mL to 40 fg/mL. The insert is the linear regression curve with over the same MMP2 range as in FIG. 7(A). FIG. 7(B) depicts a linear calibration plot of the current density vs. MMP-2 concentration range from 0.01 ng/mL to 100 ng/mL with a linear regression equation Y=33.8+8.8X, r=0.999 (n=18), P<0.0001, Sy/x=18.6.

Quantitation of MMP-2 by the DSCPO Method.

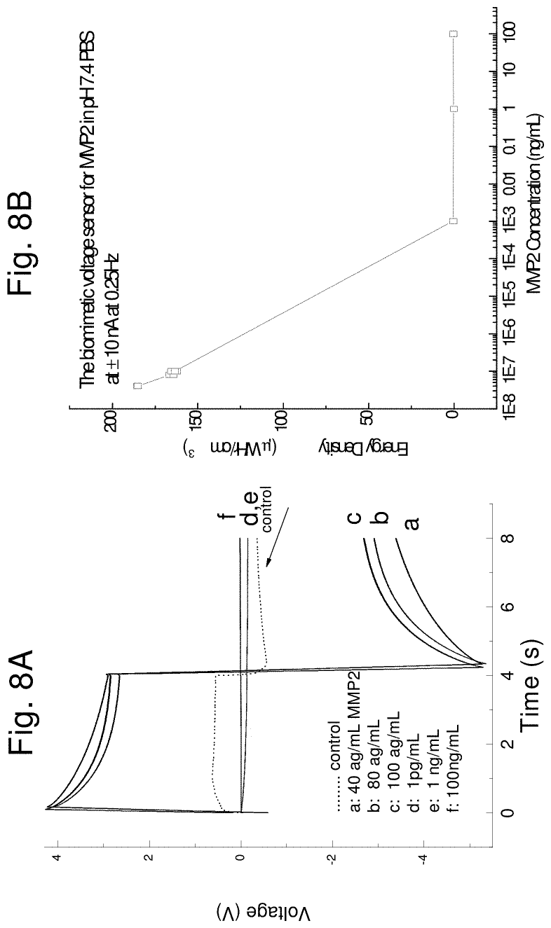

FIG. 8(A) depicts the DSCPO voltage curves vs. time at 0.25 Hz at ±10 A over 40 ag/mL to 100 ng/mL MMP-2 concentrations against the control samples with each sample run triplicates. FIG. 8 (B) depicts the volumetric energy density vs. MMP-2 concentrations, and it produced a similar impression value of 1.47% (n=18) over MMP2 concentration 40 ag/mL to 100 ng/mL over energy density between 185-0.47 μWHR/cm³.

Direct Measuring MMP-2 in NIST 965A Human Serum Specimens.

The preliminary evaluation of the method application was conducted using the CA method to measure the MMP-2. The sensor was able to directly detect MMP2 in pure NIST serum specimens in the concentrations of 81.15±0.10 ag/mL for normal, 1.13±0.0016 pg/mL for hypoglycemia and 1.4±0.0001 pg/mL in hyperglycemia serum, respectively.

### Example 5—MMP-2 Concentration Levels Affect on the Sensor Energy Density Map

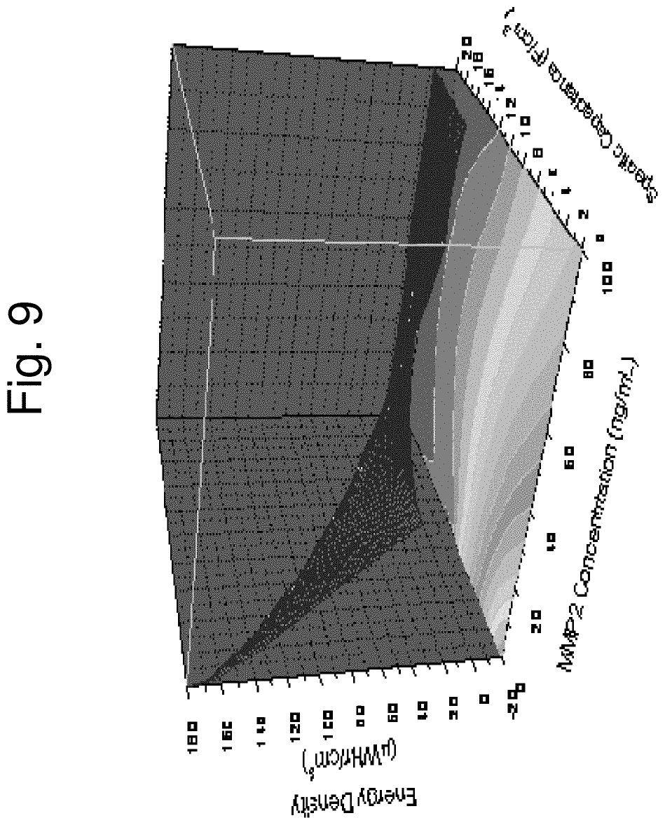

The DSCPO method was used to study the MMP-2 concentration level's affect on the sensor's energy density change related to specific capacitance change. The DSCPO results were obtained in the MMP-2 quantitation study described in the above section. The results were based on the equation of volumetric energy density, E=Cₛ·(ΔV)²/(2×3600), where Cₛ is the specific volumetric capacitance, Cₛ=[−i·Δt/ΔV]/L, Cₛ is in F/cm³ [21-22]. Δt is the time change in seconds, ΔV is the voltage change in V, i is the current in Amps, and L is the volume in cm³. FIG. 9 depicts a 3D map of the relationship between energy density of the sensor, MMP-2 concentration and specific capacitance using the voltage method. It was observed that lower MMP-2 concentration and lower specific capacitance are associated with higher energy density.

### Example 6—Comparing MMP-2 Affects on Energy Density and Capacitance

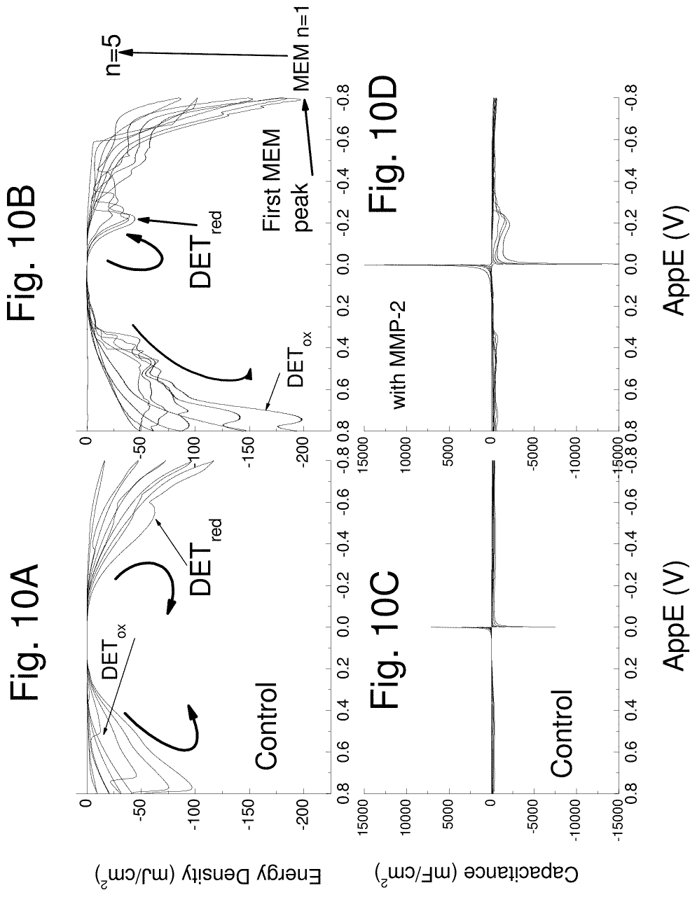

Results used for comparing MMP-2 affecting the negative energy density were presented in FIG. 10B against the controls (FIG. 10A) in three scenarios in respect to (1) the DETₒₓ peak played the role and (2) the DET_{red} peak played the role and (3) the MEM peak played a role concerning energy density and capacitance in the electrochemical potential range we studied at a fixed scan rate. FIG. 10B demonstrates the DETₒₓ peak intensity with 2-fold increased negative energy density in the presence of 40 ng/mL MMP-2 at the 5^{th} scan cycle compared with that of the control in FIG. 10A. We noticed that the significant change of energy density on MEM peak at the first scan cycle was more than a 20-fold increase for with MMP-2 than the control, where it indicates the zinc ions of MMP-2 chelating well with the COO⁻ of TCD and the two imidazole groups in the cavity of bM-β-DMCD addressed in Section 3.2.1. We also noticed that the DETₒₓ has more impact on the negative energy density with MMP-2 compared with that of DET_{red} on the negative energy density without MMP-2.

Results used for comparing MMP-2's affects on the specific capacitance of the memcapacitor were presented in FIG. 10C and FIG. 10D. FIG. 10D demonstrates that the symmetric positive and the negative capacitance peaks' values at 0.0 V (absence of an electric field) increased 3.6-fold due to the presence of MMP-2 compared to without MMP-2. The DET_{red} peak only exists at −0.2V in FIG. 10D, and played a role having increased negative capacitance 8-fold when compared to conditions without MMP-2 in FIG. 10C [33].

The hysteresis behaviors are demonstrated by the memristive/memcapacitive device in both with and without MMP-2 in the 5 scan cycles with the cross-points at zero electrochemical potential fields and zero energy density.

### CONCLUSION

We have demonstrated the advantage of the memristive/memcapacitive device with the biomimetic polarizable microtubule membrane that enables direct detection of MMP-2 with ag/mL level sensitivity in human serum specimens and the DOL reached orders of magnitude lower than published reports under antibody-free, tracer-free, and reagent-free conditions with simplified procedures by two instrumental methods. The results show a feasible application for the development of commercial fast and real-time monitoring of MMPs devices for various diseases.
[1] W. C. Parks and R. P. Mecham, Matrix metalloproteinases, Academic Press, San Diego, 1998.
[2] K. Siemianowicz, W. Likus and J. Markowski, Metalloproteinases in Brain Tumors, 2015. http://dx.doi.org/10.5772/58964.
[3] E-M. Schnaeker, R. Ossig, T. Ludwig et. al., Microtubule-dependent matrix metalloproteinases-2/matrix metalloproteinases-9 exocytosis: Prerequisite in human melanoma cell invasion, Cancer Research, 64, 8924-8931, 2004.
[4]. M. Rydlova, 1. Holubec jr., M. Ludvikova jr, D. Klfert Biological activity and clinical implications of the matrix metalloproteinases, Anticancer Research 28: 1389-1398, 2008.
[5]. F. Riedel, K. Gotte, J. Schuwalb, K. Hormann, Serum levels of matrix metalloproteinase-2 and -9 in patients with head and neck squamons cell carcinoma, Anticancer Research, 20(5 A), 3045-3049, 2000.
[6]. K. Dahiya, R. Dhankhar, Updated overview of current biomarkers in head and neck carcinoma, World J. of Methodology, 6(1), 77-86, 2016.
[7]. S. Ghargozlian, K. Svennevig, H-J Bangstad et. al., Matrix metalloproteinases in subjects with type 1 diabetes, BMC Clinical Pathology, 9(7), 2009. doi:10.1186/1472-6890-9-7.
[8]. M. Hermandez-Guillamon, S. Mawhirt, S. Blais, et. al., Sequential beta-amyloidal degradation by the matrix metalloproteinases MMP-2 and MMP-9, J. of Biol Chem, 290(24), 15078-15091, 2015.
[9]. R Wang, G Q Zeng, R Z Tong, D Zhou, Z Hong, Serum matrix metalloproteinase-2: A potential biomarker for diagnosis of epilepsy. Epilepsy Research, 122, 114-119, 2016.
[10]. R. E. Vandenbroucke, C. Libert, is there new hope for the therapeutic matrix metalloproteinase inhibition? Nature Reviews Drug Discovery, 13, 904-927, 2014.
[11]. G. Yang, L. Li, R K. Rana, J-J. Zhu, Assembled gold nanoparticles on nitrogen-doped graphene for ultrasensitive electrochemical detection of matrix metalloproteinase-2, Carbon 61, 367-366, 2013.
[12]. T. Zheng, R. Zhang, Q. Zhang et. al., Ultrasensitive dual-channel detection of matrix metalloproteinase-2 in human serum using gold-quantum dot core-satellite nanoprobes, ChemComm, 49, 7881-7883, 2016.
[13]. E. T. Chen, S-h Duh, C. Ngatchou, J. T. Thornton and P. T. Kissinger, Selectivity study for a reagent-less and enzyme-free nanopore glucose sensor against interferences from non-glucose sugars and other substances, NSTi-Nanotech (3), 101-104, 2011.
[14]. E. T. Chen, Y. Shen, C. Ngatchou, J. T. Thornton, S-h Duh, P. T. Kissinger, A nanopore biomimetic device quantitatively detects early stage cancer cells; a contour map multiple variable correlation method assesses the heat of cancer cells released, NSTi-Nanotech, 198-201, 2012.
[15]. E. T. Chen, J. T. Thornton, C. Ngatchou, S-h Duh, P. T. Kissinger, Study of the correlations between direct electron transfer rate constants and the effectiveness of cancer inhibitors using nanobiomimetic sensors, NSTi-Nanotech (3), 115-118, 2013.
[16]. S-H. Duh, J. Thornton, P. T. Kissinger and E. T. Chen, A nanobiomimetic neuronal memcapacitor serves as a voltage sensor and an amperometry sensor for reagent-less direct detection of sub pM soluble Amyloid-β. Biotech, Biomaterials and Biomedical: TechConnect Briefs, 172-175, 2015.
[17]. (a) E. T Chen, J. Thorten, C. Ngatchou, S-H Duh, Nanostructured memristor sensor mimics acetylcholinesterase (ACHE) active sites in the gorge for fM detection of acetylcholine, NSTi-Nanotech, 2, 169-172, 2014. (b) E. T. Chen, J. T. Thornton and Mulchi, Jr. C., Early forming a hummingbird-like hovering neural network circuitry pattern with reentrant spatiotemporal energy-sensory orientation privileged to avoid “epilepsy” based on a biomimetic acetylcholinesterase memcapacitor prosthesis, Sensors and Transducers Journal, 191(8), 84-99, 2015. (c). Human milk shows immunological advantages over organic milk samples for infants in the presence of Lipopolysaccharide (LPS) in 3D energy maps using an organic nanobiomimetic memristor/memcapacitor, Sensors and Transducers, 203(8), 57-68, 2016.
[18]. (a) E. T. Chen, J. Thornton and C. Mulchi Jr., Mapping circular current for a single brain cancer cell's spatial-temporal orientations based on a memristor/memcapacitor, Sensors & Transducers, 183(12), 72-83, 2014. (b) E. T. Chen, Nanobiomimetic sensing and energy storage, book in Dekker Encyclopedia of Nanoscience and Nanotechnology, S. Lyshevski, (Ed) Second Edition: Mar. 8, 2013 and (c) E. T. Chen, Nanostructure Biomimetic Sensing and Energy Storage: Organic Memristor/Memcapacitors in Dekker Encyclopedia of Nanoscience and Nanotechnology, Third Edition Taylor and Francis: New York, 2017. Published online Jan. 27, 2017. DOI: 10.1081/E-ENN3-120054061.
[19]. J. L. Malcos and W. O. Hancock, Engineering tubulin: microtubule functionalization approaches for nanoscale device applications, Appl. Microbiol Biotechnol, 90(1), 1-10, 2011.
[20]. E. T. Chen and H. L. Pardue, Analytical applications of catalytic properties of modified cyclodextrins, Anal. Chem, 65(19), 2583-2587, 1993.
[21]. M. F. El-Kady, V. Strong, S. Dubin, R. B. Kaner, Laser Scribing of High-Performance and Flexible Graphene-Based Electrochemical Capacitors, supplement materials, Science, 335, 1326-1330, 2012.
[22]. J. R. Miller, R. A. Outlaw, B. C. Holloway, Graphene double-layer capacitor with ac line-filtering performance, Science, 329, 1637-1639, 2010.
[23]. O. Kleifeld, P. E. Van den Steen, A. Frenkel et. al., Structural characterization of the catalytic active site in the latent and active natureal gelatinase B from human neutrophils, The journal of Biological Chem., 275(44), 34335-34343, 2000.
[24]. J. Pottel, E. Therrien, J. L. Gleason and N. Moitessier, Docking ligands into flexible and solvated macromolecules, 6. Development and application to the docking of HDACs and other zinc metalloenzymes inhibitors, J. of Chem information and modeling, 54, 254-265, 2014.
[25] M. D. Ventra, Y. V. Pershin, On the physical properties of memristive, memcapacitive, and meminductive systems, Nanotechnology, 24, 255201, 2013.
[26]. K. Conant, P. E. Gottschall, Matrix metalloproteinases in the central nerve system, Imperial College Press, 2005.
[27]. N. J. Clendeninn, K. Appelt, Matrix metalloproteinases inhibitors in cancer therapy, XIII, 262, A product of Humana Press, Springer, 2001.
[28]. E. D. Karagiannis, A. S. Popel, A theoretical model of type1 collagen proteolysis by matrix metalloproteinase (MMP)2 and membrane type 1 MMP in the presence of tissue inhibitor of metalloproteinase 2, The J. Biological Chemistry, 279(37), 39105-39114, 2004.

What is claimed is:

1. A direct attomolar concentration protein detecting device comprising:
an electrode comprising a substrate of gold;
a self-assembling membrane (SAM) comprising a polymer matrix comprised of an electrically conductive copolymer; wherein the copolymer is further comprised of: one or more bis imidazole substituted dimethyl-β-cyclodextrin (bM-β-DMCD) molecules, one or more β-cyclodextrin (β-CD) having at least one or more acetyl groups; one or more polyethylene glycol polymers; one or more poly(4-vinylpyridine) polymers forming the SAM having a cross-nanotube made surface structure that promotes direct electron-relay mimicking an function of antibody-Matrix Metalloproteinase (MMP) to interact with a MMP protein molecule.
1. According to claim 1, wherein the SAM further comprises hydrogen bounding or hydrophobic interaction with the TCD . . . PEG or TCD . . . PVP.
2. According to claim 1, wherein the sensor is a toroidal array dual electrochemical sensing device.
